(19) United States
(12) Patent Application Publication
(10) Pub. No.: US 2023/0226084 A1
(43) Pub. Date: Jul. 20, 2023

(54) NON-INVASIVE METHOD OF TREATING COVID-19 INFECTION

(71) Applicant: Palaniappan Viswanathan MUTHIAH, Wilayah Persekutuan (MY)

(72) Inventor: Palaniappan Viswanathan MUTHIAH, Wilayah Persekutuan (MY)

(21) Appl. No.: 17/589,732

(22) Filed: Jan. 31, 2022

(30) Foreign Application Priority Data
Jan. 19, 2022 (MY) PI 2022000330

(51) Int. Cl. A61K 31/635; A61K 31/196; A61K 31/192; A61K 31/64; A61K 31/7004; A61K 31/137; A61P 31/14

(52) U.S. Cl. CPC A61K 31/635; A61K 31/64; A61K 31/137; A61K 31/192; A61K 31/196; A61K 31/7004; A61P 31/14

(57) ABSTRACT

The present invention relates to a method for treating a Coronavirus disease 2019 (COVID-19) infected subject by reducing water content in the lungs comprising, inducing urination in a subject through urination training, administration of diuretics, or a combination of both.

### FIELD OF INVENTION

The present invention relates to methods for the treatment of the Coronavirus disease COVID-19.

### BACKGROUND OF THE INVENTION

ORIGIN of the COVID-19 or Coronavirus disease 2019 caused by the SARS-CoV-2 virus. While it has been established that most disease-causing viruses appear to have a preferred niche of an acid and relatively DRY substratum such as the throat, whereas SARS-CoV-2 virus appears to prefer a WET and acid substratum.

Various methods have been employed in order to combat COVID-19 infection such as vaccination. The purpose of any vaccination is to prompt the body to produce a defensive mechanism, so that when a virus infects, it will get destroyed. When a person is injected with a vaccine against Covid-19 Coronavirus, said vaccine has not been able to completely prevent the infection, and has also not been able to protect the morbidly obese people who may or may not have any of the non-communicable diseases, who consume increased and excessive water.

Research scientists involved in the manufacture of pharmaceutical drugs have been trying to produce drugs that could prevent or cure the COVID-19 disease. However, most of such drugs do not seem to have the capacity to dry up the accumulated fluids present in the lungs of infected subjects, hence there is a difficulty in producing a curative treatment.

Prior art WO2021216399A1 which relates to pulmonary aminosterol compositions and methods of using the same to treat microbial infections which uses lactate or dilactate salts of aminosterols administered via inhalation or pulmonary administration. Said aminosterol compositions were used to a subject diagnosed having coronavirus infection which uses furosemide in its treatments along with bronchodilators, inhaled corticosteroids, antibiotics, pulmonary surfactant, mucolytics, biologicals, genes, prostanoids, surfactants, heparin, and morphine.

Prior art WO2021211997A1 discloses methods of treating Acute Respiratory Distress Syndrome using B cells, particularly said method is for treating a chronic inflammatory disease in a subject in need thereof, said method comprising administering to the subject a poxvirus, wherein the disease is not a cancer, and is also used in treating Coronavirus diseases such as disease caused by SARS-CoV-2. WO2021211997A1 also uses diuretics in its method in order to increase urination to remove excess fluid from the body to help prevent fluid from building up in the lungs.

The following related methods of treatment in current existing technologies which rely on multiple techniques, pharmaceuticals, and methodologies that are not providing direct treatment and cure for the root cause of COVID-19 infections and accompanying complications. It is the objective of the present invention to provide a non-invasive method for treating COVID-19 infections which requires readily available materials and equipment.

### SUMMARY OF THE INVENTION

In one aspect, the invention provides a method for treating a COVID-19 infected subject by reducing water content in the lungs comprising the steps of, inducing urination in said COVID-19 infected subject through urination training, administration of diuretics, or a combination of both.

In an aspect of the invention, said COVID-19 infected subject has a severe breathing problem requiring the use of a ventilator in an Intensive Care Unit (ICU).

In an aspect of the invention, said method for treating a COVID-19 infected subject having a severe breathing problem requiring the use of a ventilator to an Intensive Care Unit (ICU) is provided. Said method comprises administering glucose saline intravenously to said subject if said subject is hypoglycemic, or water content in the lungs is reduced by making said subject to inhale warm air from a hand-held hair drier for just about 5 seconds at any one time, and repeatedly done so for 2 or 3 minutes until the breathing process returns to normal, or water content in the lungs is reduced by positioning an infrared light device (having an incandescent 150 W Infrared bulb) near the nostrils to warm up the air inhaled by said subject, or administering Salbutamol (as a metered-dose aerosol or dry powder inhaler – 90 or 100 mcg/actuation @ 1 or 2 inhalations every 4-6 hours, not exceeding 800 mcg per day, just for one or two days) or even Ventolin HFA (Albuterol inhalation – an Adrenergic bronchodilator: 2 sprays repeated every 4 to 6 hours, for 1 or 2 days), to the said subject.

In an aspect of the invention, said method for treating a COVID-19 infected subject comprises preventing the occurrence of palmer hyperhidrosis and pulmonary oedema in said COVID-19 infected subject by regulating urination frequency of said subject to be once every 2 hours while awake.

In an aspect of the invention, said method for treating a COVID-19 infected subject comprises maintaining a relative humidity (RH) of at 30% to 50% RH around the subject, preventing hypoxia in said infected subject by preventing said subject from wearing any article of protection over the face such as a facemask, preventing lymphatic fluid stagnation by preventing said subject to wear any tightly worn article of clothing around or near the abdominal and pubic areas, oral administration of urine promoters or loop diuretics selected from the group consisting of furosemide, bumetanide, ethacrynic acid, and demadex until an oral intake input of water to urine output ratio reaches 1.0 to 1.2, after drinking about 2 L or more daily. In a preferred embodiment, said tightly worn article of clothing is selected from the group consisting of tight underwear, briefs, panties, singlets, brassieres, and any similar article of clothing known to a person of ordinary skill in the art. Relative humidity may be measured using a hygrometer.

In an aspect of the invention, when said COVID-19-infected subject has a Body Mass Index (BMI) of less than 18.5, urine promoters or loop diuretics is administered to said infected subject only for once a day for 1-2 days or said infected subject’s excessive oral water intake is controlled between 2 and 2.5 L per day and urine output per day is more than 2 L. In a preferred embodiment, said method for treating a COVID-19 infected subject with BMI of less than 18.5 further comprises preventing said infected subject from wearing any article of protection over the face such as a facemask. In a preferred embodiment, said method for treating a COVID-19 infected subject with BMI of less than 18.5 further comprises drying up the lungs of said subject with the use of an infrared light, a hair drier, or an intravenous administration of diuretics.

### DETAILED DESCRIPTION OF THE INVENTION

Further understanding of the object, construction, characteristics and functions of the invention, a detailed description with reference to the embodiments is given in the following.

The present invention provides a method for treating Coronavirus disease 2019 (COVID-19), particularly a method for treating a COVID-19 infected subject by reducing water content in the lungs comprising the steps of, inducing urination in said COVID-19 infected subject through urination training, administration of diuretics, or a combination of both. In a preferred embodiment, the present invention employs urination training, and use of diuretics to reduce the water content in the lungs of a COVID-19 infected subject until the breathing of the infected subject becomes smooth and unimpeded, and also the constant cough of the infected subject stops.

In one embodiment, said COVID-19 infected subject has a severe difficulty in breathing requiring the use of a ventilator in an Intensive Care Unit (ICU).

In another embodiment, a method for treating a COVID-19 infected subject having a severe breathing problem requiring the use of a ventilator in an Intensive Care Unit (ICU) is provided. Said method comprises administering glucose saline intravenously to said subject if said subject is hypoglycemic, or water content in the lungs is reduced by making said subject to inhale warm air from a hand-held hair drier for just about 5 seconds at any one time, and repeatedly done so for 2 or 3 minutes until the breathing process returns to normal, or water content in the lungs is reduced by positioning an infrared light device near the nostrils to warm up the air inhaled by said subject, or administering Salbutamol (as a metered-dose aerosol or dry powder inhaler – 90 or 100 mcg/actuation @ 1 or 2 inhalations every 4-6 hours, not exceeding 800 mcg per day, just for one or two days) or even Ventolin HFA (Albuterol inhalation – an Adrenergic bronchodilator: 2 sprays repeated every 4 to 6 hours, for 1 or 2 days), to the said subject. The use of Salbutamol or Ventolin helps in the momentary dilation of the alveolar passages that makes breathing comfortable. This approach may have to be initiated only after the preliminary use of any ventilator for at least a short while, and repeated until the disappearance of the pulmonary oedema.

In one embodiment, said method for treating a COVID-19 infected subject comprises preventing the occurrence of palmer hyperhidrosis and pulmonary oedema in said COVID-19 infected subject by regulating urination frequency of said subject to be once every 2 hours while awake.

In one embodiment, said method for treating a COVID-19 infected subject comprises maintaining a relative humidity (RH) of at 30% to 50% RH around the subject, preventing hypoxia in said infected subject by preventing said subject from wearing any article of protection over the face such as a facemask, preventing lymphatic fluid stagnation by preventing said subject to wear any tightly worn article of clothing around or near the abdominal area, oral administration of urine promoters or loop diuretics selected from the group consisting of furosemide, bumetanide, ethacrynic acid, and demadex until an oral intake input of water to urine output ratio reaches 1.0 to 1.2. In a preferred embodiment, said tightly worn article of clothing is selected from the group consisting of tight underwear, briefs, panties, singlets, brassieres, and any similar article of clothing known to a person of ordinary skill in the art. In a preferred embodiment, furosemide is administered at a dose of 20 to 40 mg, twice daily at 6-8 hours intervals for about 3 days. In a preferred embodiment, bumetanide is orally administered on alternate days or at a dose of 0.5 to 2 mg once a day for about 3 days. In a preferred embodiment, ethacrynic acid is administered at a dose of 50 to 200 mg orally per day in 1 or 2 divided doses for a few days until smooth and unimpeded breathing in the infected subject is observed for a preferred embodiment, demadex is orally administered at a dose of 20 mg once daily until the subject’s palms, feet and armpits stop sweating, which can be measured (to be below a Relative Humidity (RH) reading of below 80%) with the use of a hygrometer whose measuring probe is held within the palm (with fingers closed) of the infected subject for 1 or 2 minutes, and this (below 80% RH at the palm) will indicate that the lungs have become dried up without the oedema that promotes Covid-19 Coronavirus infection.

In managing COVID-19 infected subjects who are either admitted in an Intensive Care Unit (ICU) or having severe difficulty in breathing, said COVID-19 infected subjects’ environment or room relative humidity (RH) is maintained slightly above 30% RH in order to dry up the water-logged lungs or excessive moisture filled lungs of the patient. COVID-19 infected subjects have pulmonary oedema which is a condition of having an increased or excessive water or moisture in the lungs. Water stagnation in the lungs promotes the infection and persistence of the virus SARS-CoV-2 within the lungs. An article of protection over the face such as face mask are prevented to be worn in COVID-19 infected subjects in order to prevent complications that could arise due to increased hypoxia. Wearing of face mask decreases the normal O₂ intake, and also increases the re-absorption of CO₂ which causes to increase the acidity of the stagnated water in the lungs. Tightly worn article of clothing such as briefs, panties, and singlets are also preferred to be not worn by said infected subjects in order to prevent lymph fluid stagnation. In profusely sweating obese people who urinate just one or twice a day, it is preferred that administration of diuretics is intravenous. The purpose of administering diuretics in the medication to COVID-19 infected subjects is to drain away the excess water that gets stored in the body (essentially in the lungs) due to habitual under-urination of the obese and overweight people. Under-urination promotes pulmonary oedema which clogs up the alveoli and causes hypoxia that often promotes respiratory problems and diseases.

In patients having severe difficulty to breathing, aside from the emergency use of mechanical ventilators in ICU-admitted patients, the natural breathing of a subject/patient may be restored using hair drier, infra-red light or any device which may be used to provide warm air for inhalation. Said warm air being inhaled would warm up the stagnated water in the lungs and enhance smooth two-way breathing. An infrared light is used for heating up the air near the nostrils.

In one embodiment, when said COVID-19-infected subject has a Body Mass Index (BMI) of less than 18.5, urine promoters or loop diuretics is administered to said infected subject at the rate of once a day for 1-2 days or said infected subject’s excessive oral water intake is controlled to be between 2 and 2.5 L per day and urine output per day is more than or equal to 2 L. In a preferred embodiment, said method for treating a COVID-19 infected subject with BMI of less than 18.5 further comprises preventing said infected subject from wearing any article of protection over the face such as a facemask. In a preferred embodiment, said method for treating a COVID-19 infected subject with BMI of less than 18.5 further comprises drying up the lungs of the said subject with the use of an infrared light, a hair drier, or an oral administration of diuretics.

When it concerns hydration management or oral intake of water, it was observed that reduced oral consumption of water at a reduced quantity of 500 ml per day did not give rise to profuse sweat, sensible perspiration or palmer hyperhidrosis as well as pulmonary oedema. As a result, such a reduced water consumption of 500 ml or less tends to make a person morbidly obese, and subjects the person only to a milder infection of the Covid-19 Coronavirus, and the infected subject remains only as an asymptomatic case. Such an infected obese subject with milder infection will have to be trained to orally consume 2 to 2.5 L daily, by a gradual increase, while at the same time such a reduced water drinking subject will have to be trained to urinate liberally at a rate of once every two hours while awake, until the water intake to urine output ratio stays at 1.0 to 1.2.

Meanwhile, subjects who drink more than 2 L of water per day, and under-urinate at the same time to only 2 or 3 times a day, it is observed that these subjects sweat profusely (have hyperhidrosis), have pulmonary oedema, and get their lung tissues acidified due to the expired CO₂ turning into carbonic acid, and thereby suffer from impeded O₂ inhalation, which in turn increases the severity of the infection and breathing suffocation.

As would be apparent to a person having ordinary skilled in the art, the aforedescribed composition may be provided in many variations, modifications or alternatives relating to a method of treating a COVID-19 infected subject. The principles and concepts disclosed herein may also be implemented in various manners which may not have been specifically described herein but which are to be understood as encompassed within the scope and letter of the following claims.

### Working Example

A subject (a medical professional, aged: 36) having confirmed of COVID-19 infection through nasopharyngeal swab (CT Value N Gene 5.62: CT Value RdRp Gene 5.62 ABBOTT M2000). This subject who was drinking more than 2.5 L water and urinating only thrice daily, had mild body odour with sweaty palms, feet and armpits, whose Relative Humidity (RH) at his palms, determined with the use of a hygrometer, measured 93%, became infected with the above virus, and developed mediocre cough, headache, body ache, fever and breathing difficulties, felt very heaty and tired with second degree fever that required the use of fast-running fans or air-conditioners even during nocturnal hours while sleeping. All the described symptoms subsided on the second day after orally taking loop diuretics furosemide twice daily, at the rate of 40 mg in the mornings, and 20 mgs with 6 hours interval for 3 days, and the subject totally recovered by night on the third day. Subsequently, the subject, upon the Inventor’s request, a while later, obtained an official Government hospital certificate, confirming his negative status for the infection. (Laboratory references: HSL/CVD/21/34750 & RTK7/CVD/21/12287).

1. A method for treating a Coronavirus disease 2019 (COVID-19) infected subject by reducing water content in the lungs comprising:
inducing urination in a subject through urination training, administration of diuretics, or a combination of both.

2. The method for treating a COVID-19 infected subject of claim 1, wherein, the said COVID-19 infected subject is a person with severe breathing difficulty necessitating the use of a ventilator at an Intensive Care Unit (ICU) in a hospital.

3. The method for treating a COVID-19-infected subject of claim 1, comprising:
maintaining a relative humidity (RH) of 30% to 50% RH around the subject;
preventing hypoxia in said infected subject by preventing the said subject from wearing any article of protection over the face such as a facemask;
preventing lymphatic fluid stagnation by preventing said subject to wear any tightly worn article of clothing around or near the abdominal or pubic area; and
oral administration of urine promoters or loop diuretics selected from the group consisting of furosemide, bumetanide, ethacrynic acid, and demadex until a oral intake of water to urine output ratio reaches 1.0 to 1.2.

4. The method for treating a COVID-19-infected subject of claim 1, wherein when said COVID-19-infected subject has a Body Mass Index (BMI) of less than 18.5:
urine promoter or loop diuretics is administered to the said infected subject only once a day for 1-2 days, or
said infected subject’s excessive oral water intake is controlled between 2 and 2.5 L per day and urine output per day is 2 L or more.

5. The method for treating a COVID-19-infected subject of claim 4, further comprising preventing said subject from wearing any article of protection over the face such as a facemask.

6. The method for treating a COVID-19-infected subject of claim 4, further comprising drying up the lungs of the said subject with the use of an infrared light, a hair drier, or an intravenous administration of diuretics.

7. The method for treating a COVID-19-infected subject of claim 3, wherein said article of clothing is selected from the group consisting of tight underwear, briefs, panties, singlets, brassieres, and any similar article of clothing.

8. The method for treating a COVID-19-infected subject of claim 2, further comprising:
administering glucose saline intravenously to the said subject if the said subject is hypoglycemic; or
water content in the lungs is reduced by making said subject to inhale warm air from a hand-held hair drier for just about 5 seconds at any one time, and repeatedly done so for 2 or 3 minutes until the breathing process returns to normal, or
water content in the lungs is reduced by positioning an infrared light device near the nostrils to warm up the air inhaled by the said subject; or
administering salbutamol or ventolin to the said subject.

9. The method for treating a COVID-19-infected subject of claim 2, further comprising:
controlling water intake in the said subject by gradually adjusting the oral water intake of a subject to a 2000 ml to 2500 ml level per day.

10. The method for treating a COVID-19-infected subject of claim 1 comprising:
preventing the occurrence of palmer hyperhidrosis and pulmonary oedema in the said COVID-19 infected subject by regulating urination frequency of the said subject to be once every 2 hours while awake.

11. The method for treating a COVID-19-infected subject of claim 2, comprising:
maintaining a relative humidity (RH) of 30% to 50% RH around the subject;
preventing hypoxia in said infected subject by preventing the said subject from wearing any article of protection over the face such as a facemask;
preventing lymphatic fluid stagnation by preventing said subject to wear any tightly worn article of clothing around or near the abdominal or pubic area; and
oral administration of urine promoters or loop diuretics selected from the group consisting of furosemide, bumetanide, ethacrynic acid, and demadex until a oral intake of water to urine output ratio reaches 1.0 to 1.2.

12. The method for treating a COVID-19-infected subject of claim 2, wherein when said COVID-19-infected subject has a Body Mass Index (BMI) of less than 18.5:
urine promoter or loop diuretics is administered to the said infected subject only once a day for 1-2 days, or
said infected subject’s excessive oral water intake is controlled between 2 and 2.5 L per day and urine output per day is 2 L or more.

13. The method for treating a COVID-19-infected subject of claim 12, further comprising preventing said subject from wearing any article of protection over the face such as a facemask.

14. The method for treating a COVID-19-infected subject of claim 12, further comprising drying up the lungs of the said subject with the use of an infrared light, a hair drier, or an intravenous administration of diuretics.

15. The method for treating a COVID-19-infected subject of claim 11, wherein said article of clothing is selected from the group consisting of tight underwear, briefs, panties, singlets, brassieres, and any similar article of clothing.
